(19) United States
(12) Patent Application Publication
(10) Pub. No.: US 2020/0376287 A1
(43) Pub. Date: Dec. 3, 2020

(54) METHODS FOR INCREASING TGF-B SIGNALING

(71) Applicant: TECHNION RESEARCH & DEVELOPMENT FOUNDATION LIMITED, Haifa (IL)

(72) Inventors: Asya ROLLS, Tel Aviv (IL); Maria KROT, Haifa (IL); Hilla AZULAY-DEBBY, Haifa (IL)

(21) Appl. No.: 16/885,696

(22) Filed: May 28, 2020

Related U.S. Application Data
(60) Provisional application No. 62/953,400, filed on Dec. 24, 2019.
(60) Provisional application No. 62/853,868, filed on May 29, 2019.

(51) Int. Cl. A61N 2/00; C07K 14/495

(52) U.S. Cl. CPC A61N 2/002; C07K 14/495

(57) ABSTRACT

The present invention, in some embodiments thereof, is directed to a method for preserving or promoting oral tolerance in a subject in need thereof, including modulating neurons in the mid-posterior region of the insular cortex (mpIC). Further provided is a method for increasing TGF-β signaling in a subject in need thereof.

### CROSS-REFERENCE TO RELATED APPLICATIONS

This application claims the benefit of priority of U.S. Provisional Patent Application No. 62/853,868 titled “EFFECTS OF FOOD-RELATED SENSORY INFORMATION ON THE INTESTINAL IMMUNE SYSTEM”, filed May 29, 2019, and of U.S. Provisional Patent Application No. 62/953,400 titled “METHODS FOR INCREASING TGF-B SIGNALING”, filed Dec. 24, 2019, the contents of which are incorporated herein by reference in their entirety.

### FIELD OF THE INVENTION

The present invention, in some embodiments thereof, is in the field of neuroimmunology and neuromodulation.

### BACKGROUND OF THE INVENTION

Immune cells in the gut are exposed to a high number of foreign antigens, on a daily basis. Generally, the primary function of the immune system is to protect the host from pathogens, thus it is designed to identify and eliminate foreign antigens by eliciting an inflammatory response. However, as most of the ingested substances, such as food antigens and commensals, are innocuous, tolerogenic environment must be maintained in the gut in order to prevent pathological immune reactivity. Therefore, the immune response towards orally administrated antigens suppressed by an active process, is termed oral tolerance. Oral tolerance is defined as the local and systemic hyporesponsiveness to a subsequent challenge that occurs when exogenous antigens are administered by the oral route. The establishment of tolerance to food antigens is critical since when it breaks down its results in various pathologies, such as coeliac disease and/or food allergies.

Development of oral tolerance to food antigens involves early adjustments in the intestinal mucosa. A local tolerogenic environment is conditioned by cytokines, such as Interleukin 10 (IL-10) and Transformation growth factor beta (TGF-β), providing a non-specific inflammation control. For example, TGF-β signaling is shown to be involved in the local differentiation of CD103 integrin-expressing dendritic cells (DCs) that hold tolerogenic properties. These specialized DCs migrate to the draining mesenteric lymph nodes (mLN) following exposure to food antigens in the mucosal lamina propria and produce TGF-β and retinoic acid (RA) while presenting the antigens to naïve CD4+ T cells. The high levels of TGF-β and RA promote the Foxp3 synthesis by the naïve CD4+ T cells and their differentiation into antigen-specific regulatory T cells (Treg) that utilize various sets of mechanisms to maintain tolerance. Although the process of Treg differentiation and mechanisms of suppression are well studied, the factors that initially drive the formation of tolerogenic environment in the gut (e.g. TGF-β secretion) remain unknown.

Here, we suggest the involvement of gut-brain communication in the regulation of the intestinal environment and oral tolerance establishment. Sensory information (e.g. taste, smell) that acquired by the brain during the ingestion of potential food can be valuable to the immune system, as it can allow one to determine whether the consumed food is nourishing or if it potentially contains harmful substances (e.g. toxins or pathogens) and thus requires the induction of a protective immune response. However, it is unclear whether external sensory information encoded by the brain and predicts the quality of the food can be provided to immune cells in the gut or to affect their activity.

### SUMMARY OF THE INVENTION

According to one aspect, there is provided a method for preserving or promoting oral tolerance in a subject in need thereof, comprising the step of modulating neurons in the mid-posterior region of the insular cortex (mpIC) of the subject, thereby preserving or promoting oral tolerance in the subject.

According to another aspect, there is provided a method for increasing TGF-β signaling in a subject in need thereof, comprising a step selected from: (a) inhibiting neurons of the agranular insula of the subject, (b) activating neurons of the: dysgranular insula of the subject, granular insula of the subject, or both, or (c) a combination of (a) and (b); thereby increasing TGF-β signaling in the subject.

In some embodiments, modulating comprises: (a) inhibiting neurons of the agranular insula of the subject, (b) activating neurons of the: dysgranular insula of the subject, granular insula of the subject, or both, or (a) and (b).

In some embodiments, modulating comprises a step of applying a non-invasive brain stimulation (NIBS) to the subject.

In some embodiments, the NIBS is selected from neurofeedback or magnetic stimulation (MS).

In some embodiments, preserving or promoting oral tolerance comprises increasing the activity, the abundance, or both, of at least one cell selected from the group consisting of: a CD11b+CD11c+ myeloid cell, CD11b+CD11c− myeloid cell, a CD11b-CD11c+ myeloid cell, a Foxp3+CD25+CD4TCRβ T cell, a Foxp3+CD25+CD8TCRβ T cell, and a EpCAM+CD45− epithelial cell.

In some embodiments, the increased activity, abundance, or both, comprises increased transformation growth factor beta (TGF-β) signaling in the at least one cell.

In some embodiments, preserving or promoting oral tolerance is increasing the number of: (i) TGF-β expressing CD11b+CD11c− myeloid cells, TGF-β expressing CD11b+CD11c+ myeloid cells, TGF-β expressing CD11b−CD11c+ myeloid cells, or any combination thereof; (iii) TGF-β expressing EpCAM+CD45− epithelial cells; (iv) TGF-β expressing Foxp3+CD25+CD4TCRβ cells, TGF-β expressing Foxp3+CD25+CD8TCRβ cells, or both, and any combination of (i) to (iv), in at least one tissue of the subject, wherein the tissue is selected from the group consisting of: mesenteric lymph node (mLN), the lamina propria (LP) of the small intestine, and the intraepithelial layer (IEL) of the small intestine.

In some embodiments, the method further comprises a step of determining an increased activity, abundance, or both, of at least one cell selected from the group consisting of: a CD11b+CD11c− myeloid cell, a CD11b+CD11c+ myeloid cell, a CD11b−CD11c+ myeloid cell, a Foxp3+CD25+CD4TCRβ cell, a Foxp3+CD25+CD8TCRβ cell, a EpCAM+CD45− epithelial cell, and any combination thereof, in a sample obtained or derived from the subject.

In some embodiments, the method further comprises a step of determining increased TGF-β signaling in at least one cell selected from the group consisting of: CD11b+CD11c− myeloid cell, CD11b+CD11c+ myeloid cell, CD11b−CD11c+ myeloid cell, Foxp3+CD25+CD4TCRβ cell, Foxp3+CD25+CD8TCRβ cell, EpCAM+CD45− epithelial cell, and any combination thereof, in a sample obtained or derived from the subject.

In some embodiments, increasing TGF-β signaling comprises a step of applying a non-invasive brain stimulation (NIBS) to the subject.

In some embodiments, increasing TGF-β signaling is in at least one cell selected from the group consisting of: a CD11b+CD11c+ myeloid cell, CD11b+CD11c− myeloid cell, a CD11b− CD11c+ myeloid cell, a Foxp3+CD25+CD4TCRβ T cell, a Foxp3+CD25+CD8TCRβ T cell, and a EpCAM+CD45− epithelial cell.

In some embodiments, increasing is in at least one tissue of the subject selected from the group consisting of: mesenteric lymph node (mLN), the lamina propria (LP) of the small intestine, the intraepithelial layer (IEL) of the small intestine.

In some embodiments, the subject is afflicted with an immune-associated disease.

In some embodiments, the immune-associated disease comprises any one of an autoimmune disease and a food-induced immune disease.

In some embodiments, the autoimmune disease is an inflammatory bowel diseases (IBD).

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIGS. 1A-1D include fluorescent micrographs, a graph, and location maps, showing that neurons in the mid-posterior insular cortex (mpIC) respond to oral consumption of ovalbumin (OVA). (1A) Micrographs demonstrating Arc expression in the mpIC of Arc-dVenus reporter mice that were exposed to water (left panel) or OVA (right panel). The expression of Arc was evaluated by immunohistochemical staining for GFP (green) to amplify the signal of dVenus and co-localized with DAPI nuclear staining (blue). Dotted lines are based on Allen Brain Atlas and depict the cytoarchitectonic layers of the insular cortex (granular insula; GI, dysgranular insula; DI, agranular insula; AI) and the claustrum (CI). Scale bar, 200 μm. (1B) A graph showing manual quantification of Arc-positive cells that were identified by GFP and DAPI double-positive staining per region of interest (ROI=605 mm²) in mice that were exposed to water or OVA. Student's t-test; mean±s.e.m; n=3, 4; **P<0.01. (1C) Maps showing the location of the insular cortex in mice (upper left). Arc-expressing neurons following oral consumption of OVA were found in the mid-posterior region of the insula (upper right) that comprises both gustatory and visceral cortices (lower panel). (1D) A location map and micrographs showing validation of the injection site in the mpIC. Representative image describing the expression of the inhibiting form of designer receptors exclusively activated by designer drugs (DREADDs; Gi) in neurons following stereotactic injection of a viral vector (AAV8-hSyn-hM4Di (Gi)-mCherry), in the GI and DI layers of the mpIC ((0.14-0.18) mm Anterior-Posterior (AP); (3.6-3.8) mm Medial-Lateral (ML); (2.5-2.6) mm Dorsal-Ventral (DV)).

FIGS. 2A-2E include illustration of a non-limiting experimental design and graphs showing that inhibition of neuronal activity in the mpIC impairs oral tolerance. (2A) A schematic non-limiting representation of the experimental design for two separate experiments. The upper panel describes mice that were subjected to the delayed-type hypersensitivity (DTH) model following oral consumption of water (blue marks in 2B) or OVA (50 mg/ml; yellow marks in 2B). The lower panel describes mice that were subjected to the DTH model following oral consumption of OVA, while the neuronal activity in the mpIC was unaffected (control; grey marks in 2B) or inhibited (Gi; red marks in 2B). (2B) A graph showing the change in swelling (mm²) at the area of antigen deposition, indicated by the fold of the delta between the right (R; OVA injected) and left (L; PBS injected) foot for groups from both experiments. (2C) Enzyme-linked immunosorbent assay (ELISA) results show the fold change of interferon gamma (IFNγ) levels in plasma for both experiments. (2D) A graph showing the results of a carboxyfluorescein succinimidyl ester (CFSE) proliferation assay. Flow cytometric data shows the fold change in the percent of proliferating live cells for both experiments, indicated by the ratio of CFSE^{low} cells in OVA-stimulated to CFSE^{low} cells in non-stimulated splenocytes 72 hours post-challenge ex vivo. (2E) Graphs of ELISA results showing the levels of IFN-γ (left) and interleukin 2 (IL-2; right) in the supernatant 72 h post-challenge with OVA ex vivo from both experiments. Student's t-test; mean±s.e.m; n=6, 6; *P<0.05, ***P<0.001, **** P<0.0001.

FIGS. 3A-3D are illustration of a non-limiting experimental design and graphs showing that inhibition of neuronal activity in the mpIC affects immune cells in the gut and mesenteric lymph nodes (mLNs). (3A) A schematic non-limiting representation of the experimental design. (2B-2C) Graphs of flow cytometric data analysis showing the change in percent of transformation growth factor beta (TGFβ)-expressing immune cells (CD45+; left), CD4+ and CD8+ T cells (center) and myeloid cell subpopulations (right) in the mLNs (3B) and the lamina propria (LP; 3C) of the small intestine (SI) of mpIC-inhibited mice compared to control. (3D) Graphs show the fold change in the proportion of epithelial cells (EpCAM+CD45+/−) and immune cells (EpCAM-CD45+) in the intraepithelial layer (IEL) of the SI, which express TGF-β (left) and IFNγ (center). The left panel depicts the fold change in the percent of CD69+ activated CD4+ and CD8+ T cells in the IEL Student's t-test; mean±s.e.m; n=5, 6; *P<0.05, **P<0.01, ***P<0.001.

FIGS. 4A-4D include illustration of a non-limiting experimental design and graphs showing the boosting of oral tolerance through activation of neurons in the mpIC. (4A) A schematic non-limiting representation of the experimental design. (4B-4C) Graphs of flow cytometric data analysis showing the change in percent of TGFβ-expressing immune cells (CD45+; left), CD4+ and CD8+ T cells (center) and myeloid cell subpopulations (right) in mLNs (4B) and LP (4C) of the SI of mpIC-activated mice compared to control. (4D) Graphs show the fold change in the proportion of epithelial cells (EpCAM+CD45+/−) and immune cells (EpCAM-CD45+) in the IEL of the SI that express TGF-β (left) and IFNγ (center). The left panel depicts the fold change in the percent of CD69+ activated CD4+ and CD8+ T cells in the IEL. Student's t-test; mean±s.e.m; n=4, 5; *P<0.05.

FIGS. 5A-5F include fluorescent micrographs, graphs, and location maps, showing representation of aversive information in the mpIC. (5A) Images demonstrate Arc expression in the mpIC of Arc-dVenus reporter mice that were exposed to water (left panel) or denatonium benzoate (6 mM; right panel). The expression of Arc was evaluated as described in FIG. 1A. Dotted lines are based on Allen Brain Atlas, and depict the cytoarchitectonic layers of the insular cortex (granular insula; GI, dysgranular insula; DI, agranular insula; AI) and the claustrum (CI). Scale bar, 200 μm. (5B) Manual quantification of Arc-positive cells in the region of interest (ROI=605 mm²) was performed as described in FIG. 1A in mice that consumed water or denatonium benzoate. Student's t-test; mean±s.e.m; n=3, 5; *P<0.05. (5C) The images show Arc expression in the mpIC of Arc-dVenus reporter mice that were exposed to OVA (left panel) or denatonium benzoate (right panel). The numbers (1-6) represent the layers of the cerebral cortex. Scale bar, 200 μm. (5D) Graphs represent the difference in total Arc expression (left) and the distribution of Arc-positive cells in the 3 cytoarchitectonic layers of the insular cortex (GI, DI, and AI) following exposure of mice to OVA or denatonium benzoate. (5E) The graph shows the difference in the ratio of Arc expression between the DI and AI layers of OVA- and bitter-consuming mice. (5F) Graphs depict the total difference in Arc-positive cells in the ⅔ layer of the cerebral cortex (left) of the mpIC and expression of Arc in this layer divided by the insular layers (GI, DI, and AI). Student's t-test; mean±s.e.m; n=3, 5; *P<0.05, **P<0.01.

FIGS. 6A-6D include illustration of a non-limiting experimental design and graphs showing that activation of the AI layer of mpIC results in anti-tolerogenic effects. (6A) A schematic non-limiting representation of the experimental design. (6B-6C) Graphs of flow cytometric data analysis showing the change in percent of TGF-β-expressing immune cells (CD45+; left), CD4+ and CD8+ T cells (center) and myeloid cell subpopulations (right) in mLNs (6B) and LP (6C) of the SI of mpIC-activated mice compared to control. (6D) Graphs show the fold change in the proportion of epithelial cells (EpCAM+CD45+/−) and immune cells (EpCAM-CD45+) in the IEL of the SI that express TGF-β (left) and IFNγ (center). Left panel depicts the fold change in the percent of CD69+ activated CD4+ and CD8+ T cells in the IEL. Student's t-test; mean±s.e.m; n=8, 7; *P<0.05, ****P<0.0001.

FIGS. 7A-7D include illustration of a non-limiting experimental design and graphs showing that activation of the AI layer of the mpIC during exposure to a novel innocuous antigen results in anti-tolerogenic effects. (7A) A schematic non-limiting representation of the experimental design. (7B-7C) Graphs of flow cytometric data analysis showing the change in percent of TGF-β-expressing immune cells (CD45+; left), CD4+ and CD8+ T cells (center) and myeloid cell subpopulations (right) in mLNs (7B) and LP (7C) of the SI of mpIC-activated mice compared to control. (7D) Graphs show the fold change in the proportion of epithelial cells (CD45-) and immune cells (CD45+) in the IEL of the SI that express TGF-β (left). Right panel depicts the fold change in the percent of CD69+ activated CD4+ and CD8+ T cells in the IEL. Student's t-test; mean±s.e.m; n=8, 8; *P<0.05, **P<0.01, ***P<0.001, ****P<0.0001.

FIGS. 8A-8D include illustration of a non-limiting experimental design and graphs showing the boosting of oral tolerance through inhibition of neurons in the AI layer of the mpIC. (8A) A schematic representation of the experimental design. (8B-8C) Graphs of flow cytometric data analysis showing the change in percent of TGF-β-expressing immune cells (CD45+; left), CD4+ and CD8+ T cells (center) and myeloid cell subpopulations (right) in mLNs (8B) and LP (8C) of the SI of mpIC-activated mice compared to control. (8D) Graphs show the fold change in the proportion of epithelial cells (CD4-) and immune cells (CD45+) in the IEL of the SI that express TGF-β (left). Right panel depicts the fold change in the percent of CD69+ activated CD4+ and CD8+ T cells in the IEL Student's t-test; mean±s.e.m; n=8, 8; *P<0.05, **P<0.01.

FIGS. 9A-9E include graphs showing CD69 expression on T cell populations in the IEL. (9A-9E) Graphs of flow cytometric data analysis showing the difference in the median fluorescent intensity (MFI) of the activation marker CD69 on CD4+ and CD8+ T cell populations in the IEL of the SI through the different manipulations performed: inhibition of OVA-driven activity in the GI and DI layer of the mpIC (9A), activation of the GI and DI layer of the mpIC while mice consume water (9B), activation of the AI layer of the mpIC while mice consume water (9C), activation of the AI layer of the mpIC while mice consume OVA (9D), and inhibition of the AI layer of the mpIC while mice consume OVA (9E). Student's t-test; mean±s.e.m; *P<0.05.

FIGS. 10A-10D include graphs showing TGF-β and CD69 expression on T cell populations in the mLNs. (10A-10B) Graphs of flow cytometric data analysis showing the difference in the median fluorescent intensity (MFI) of TGF-β (10A) and CD69 (10B) on CD4+ and CD8+ T cell populations in mLNs between mpIC-activated mice (GI and DI layers) and the control group. (10C-10D) Graphs represent the change in the MFI of TGFβ (10C) and CD69 (10D) on CD4+ and CD8+ T cell populations in mLNs between mpIC-inhibited (AI layer) and the control group. Student's t-test; mean±s.e.m; *P<0.05, **P<0.01, ***P<0.001, ****P<0.0001.

FIG. 11 is a heatmap showing TGF-β expression on myeloid populations in the mLNs. Heatmap showing the percentage fold change in TGF-β-expression on different subpopulations of myeloid cells (X-axis) expressing various combinations of immune markers (Y-axis) in the mLNs of mice that gone through activation while consuming water (Gq; left) or inhibition while consuming OVA (Gi; right) at the GI and DI layers of the mpIC. The activation of the GI and DI layers of the mpIC in mice exposed to water affected mainly CX3CR1+ myeloid cells in mLNs. In contrast, inhibition of the same layers in the mpIC of mice that were exposed to OVA had a broader effect. Specifically, it reduced the proportion of both TGF-β-expressing CX3CR1+ and CX3CR1− cells. For heatmap analysis P values were set with the two-stage linear step-up procedure of Benjamini, Krieger, and Yekutieli multiply correction (Q=5% for comparisons with *P<0.0292; Q=1% for comparisons with ** P<0.0043 and ***P<0.0007).

FIG. 12 includes fluorescent micrographs showing the targeting of the agranular layer of the mpIC. The images represent a validation of the injection site in the granular (GI) and dysgranular (DI) layers (left; as in FIGS. 1-4; (0.14-0.18) mm Anterior-Posterior (AP); (3.6-3.8) mm Medial-Lateral (ML); (2.5-2.6) mm Dorsal-Ventral (DV)) and in the agranular (AI) layer of the mpIC (right; as in FIGS. 6-8 (0.14-0.18) mm Anterior-Posterior (AP); (3.8-4) mm Medial-Lateral (ML); (2.7-2.8) mm Dorsal-Ventral (DV)).

FIGS. 13A-13C include graphs showing TGF-β expression on T cell populations in the LP. (13A-13C) Graphs of flow cytometric data analysis showing the difference in the median fluorescent intensity (MFI) of TGF-β on CD4+ and CD8+ T cell populations in LP of the SI through the different manipulations performed in mice that were expressing DREADDs in the agranular layer (AI) of the mpIC: activation while mice consume water (13A), activation while mice consume OVA (13B), and inhibition while mice consume OVA (13C). Student's t-test; mean s.e.m; *P<0.05, **P<0.01.

### DETAILED DESCRIPTION OF THE INVENTION

In some embodiments, the present invention is directed to a method for preserving or promoting oral tolerance in a subject in need thereof.

In some embodiments, the present invention is directed to a method for increasing transformation growth factor beta (TGF-β) signaling in a subject in need thereof.

As used herein, the phrase “oral tolerance” is defined as the local and systemic hyporesponsiveness to subsequent challenge that occurs when exogenous antigens and/or allergens are administered by the oral route. The establishment of tolerance to food antigens and/or allergens is critical since when it breaks down it results in various pathologies, such as coeliac disease and food allergies. In some embodiments, oral tolerance is an immune response. In some embodiments, an immune response comprises oral tolerance.

As used herein, “TGF-β signaling” refers to any one of: expression of the TGF-β-encoding gene, TGF-β protein synthesis, the release or secretion of mature TGF-β from cells, e.g., immune cells such as regulatory T lymphocytes (Tregs), epithelial cells, etc., the binding of mature TGF-β to a TGF-β receptor, TGF-β activity and/or the activation of molecules from the TGF-β receptor signaling pathway, or any combination thereof.

In some embodiments, the method comprises the step of modulating neurons in the mid-posterior region of the insular cortex (mpIC) of the subject.

In another embodiment, the term “modulating” is altering. In another embodiment, the term “modulating” is activating. In another embodiment, the term “modulating” is inhibiting. In another embodiment, the term “modulating” is increasing. In another embodiment, the term “modulating” is inducing. In another embodiment, the term “modulating” is elevating. In another embodiment, the term “modulating” is reducing. In another embodiment, the term “modulating” is differentially activating. In another embodiment, the term “modulating” is decreasing. In another embodiment, the term “modulating” is differentially inhibiting. In another embodiment, modulating neurons of the mpIC provides activation and/or induction of certain immune cells or sub-sets of immune cells. In another embodiment, modulating neurons of the mpIC provides inhibition of certain immune cells or particular sub-sets of immune cells. In another embodiment, modulating neurons of the mpIC provides activation and/or induction of certain immune cells or sub-sets, while at the same time provides inhibition of other immune cells or particular sub-sets of immune cells.

In some embodiments, the method comprises: (a) inhibiting neurons of the agranular insula of the subject, (b) activating neurons of the: dysgranular insula of the subject, granular insula of the subject, or both, or (a) and (b).

According to another embodiment, the method of the invention comprises inducing or maintaining an oral tolerance response in a subject, by co-activating neurons in the DI and GI of the mpIC in the subject by applying a neural modulating stimulus, e.g., neurofeedback or TMS.

According to another embodiment, the method of the invention comprises inducing or maintaining an oral tolerance response in a subject, by inhibiting neurons in the AI of the mpIC in the subject by applying a neural modulating stimulus, e.g., neurofeedback or TMS.

In some embodiments, mpIC co-activation (e.g., DI and GI) results in enhanced oral tolerance response compared to a single activation (e.g., DI, or GI). In some embodiments, mpIC co-activation (e.g., DI and GI) results in comparable enhancement of oral tolerance compared to a single activation (e.g., DI, or GI). In some embodiments, mpIC co-activation (e.g., DI and GI) results in synergistically enhanced oral tolerance compared to a single activation (e.g., DI, or GI).

In some embodiments, modulating comprises a step of applying a non-invasive brain stimulation (NIBS) to the subject.

In some embodiments, the NIBS is selected from: neurofeedback, magnetic stimulation (MS), transcranial MS (TMS), repetitive TMS (rTMS), deep TMS, cranial electrotherapy stimulation (CES), transcranial direct current stimulation (tDCS), transcranial random noise stimulation (tRNS), electroconvulsive therapy (ECT), reduced impedance non-invasive cortical electrostimulation (RINCE), or any combination thereof.

In some embodiments, NIBS is selected from neurofeedback and magnetic stimulation (MS).

In some embodiment, any NIBS modality is applicable as long as it: (a) inhibits neurons of the agranular insula of the subject, (b) activates neurons of the: dysgranular insula of the subject, granular insula of the subject, or both, or (a) and (b).

In some embodiments, the method comprises preserving oral tolerance. In some embodiments, the method comprises promoting oral tolerance. In some embodiments, the method comprises increasing oral tolerance. In some embodiments, the method comprises introducing oral tolerance to a subject, wherein the subject is devoid of oral tolerance prior to being treated according to the method of the invention. In some embodiments, the subject is characterized by having a reduced, a minimal, a partial, or any combination or equivalent thereof, oral tolerance.

As used herein, the term “preserving” comprises maintaining an existing situation, status, or level.

In some embodiments, an immune response, e.g., oral tolerance, comprises any response taken by the body to defend itself from pathogens or abnormalities. In one embodiment, an immune response comprises a response mediated or involving an immune cell. In some embodiments, an immune response comprises a response induced by antigens or allergens taken orally by the body. In some embodiments, healthy subject manifests or is characterized by an oral tolerance response comprising an increased activity, amount, or both, of TGF-β signaling, Treg, or both. In some embodiments, a subject in need thereof, manifests, comprises, or is characterized by an impaired oral tolerance comprising a reduced activity, amount, or both, of TGF-β signaling, regulatory T lymphocytes (Treg), or both.

In one embodiment, an immune response comprises any response activating or inhibiting the immune system or mediators of the immune system. In another embodiment, activation of an immune response comprises activation of an immune cell. In another embodiment, activation of an immune cell results in the proliferation of a sub-set of immune cells. In another embodiment, activation of an immune cell results in increased secretion of an immunologic mediator by the activated cell. In another embodiment, activation of an immune cell results in the engulfment and/or destruction of a pathogen, a foreign cell, a diseased cell, a host cell, a molecule derived or secreted therefrom, or any combination thereof. In another embodiment, activation of an immune cell results in the engulfment and or destruction of a neighboring cell, such as, but not limited to, a cell infected by a virus. In another embodiment, activation of an immune cell results in activating the secretion of antibodies directed to a certain molecule, epitope, pathogen, or any combination thereof. In some embodiments, an immune response is an autoimmune response, e.g., comprising any response wherein the body's immune system targets cells and/or tissues of the body. In some embodiments, an autoimmune disease is induced by food or orally encountered and/or consumed allergens. In some embodiments, an autoimmune disease comprises the production of autoantibodies.

As used herein, an immune response is any response activating any one of: myeloid cells, T-cells, epithelial cells, or any combination thereof. In another embodiment, a response activating a cell as described herein, results in: proliferation of the cell or another immune cell, secretion of immune mediators, such as cytokines, migration of an immune cell, activation of an immune cascade, or any combination thereof.

In another embodiment, an immune response is associated with a disease and a method as described herein is used to modulate the immune response, e.g., maintain or promote oral tolerance.

In some embodiments, the method comprises increasing the activity, the abundance, or both, of at least one cell selected from: a CD11b+CD11c+ myeloid cell, CD11b+CD11c− myeloid cell, a CD11b−CD11c+ myeloid cell, a Foxp3+CD25+CD4TCRβ T cell, a Foxp3+CD25+CD8TCRβ T cell, a EpCAM+CD45− epithelial cell. In some embodiments, the method comprises increasing the activity, the abundance, or both, of a plurality or a combination of cell types selected from: a CD11b+CD11c+ myeloid cell, CD11b+CD11c− myeloid cell, a CD11b−CD11c+ myeloid cell, a Foxp3+CD25+CD4TCRβ T cell, a Foxp3+CD25+CD8TCRβ T cell, a EpCAM+CD45− epithelial cell.

In some embodiments, the method comprises increasing the activity, the abundance, or both, of an immune cell. In some embodiments, the immune cell is a CD45+ immune cell.

In some embodiments, increased activity, abundance, or both, comprises increased TGF-β signaling in at least one of the herein disclosed cells.

In some embodiments, preserving or promoting oral tolerance is increasing TGF-β signaling in at least one of the herein disclosed cells.

In some embodiments, preserving or promoting oral tolerance is increasing TGF-β signaling in at least one of the herein disclosed cells, in at least one tissue of a subject. In some embodiments, a tissue is selected from: mesenteric lymph node (mLN), the lamina propria (LP) of the small intestine (SI), and the intraepithelial layer (IEL) of the SI.

In some embodiments, preserving or promoting oral tolerance is increasing the number of: (i) TGF-β expressing CD11b+CD11c− myeloid cells, TGF-β expressing CD11b+CD11c+ myeloid cells, TGF-β expressing CD11b−CD11c+ myeloid cells, or any combination thereof; (iii) TGF-β expressing EpCAM+CD45− epithelial cells; (iv) TGF-β expressing Foxp3+CD25+CD4TCRβ cells, TGF-β expressing Foxp3+CD25+CD8TCRβ cells, or both, or any combination of (i) to (iv), in at least one tissue of the subject, wherein the tissue is selected from: mLN, the LP of the SI, and the IEL of the SI.

In some embodiments, the method further comprises a step of determining an increased activity, abundance, or both, of at least one cell selected from: a CD11b+CD11c− myeloid cell, a CD11b+CD11c+ myeloid cell, a CD11b−CD11c+ myeloid cell, a Foxp3+CD25+CD4TCRβ cell, a Foxp3+CD25+CD8TCRβ cell, a EpCAM+CD45− epithelial cell, or any combination thereof, in a sample obtained from the subject.

In some embodiments, the method further comprises a step of determining increased TGF-β signaling in at least one cell selected from: CD11b+CD11c− myeloid cell, CD11b+CD11c+ myeloid cell, CD11b−CD11c+ myeloid cell, Foxp3+CD25+CD4TCRβ cell, Foxp3+CD25+CD8TCRβ cell, EpCAM+CD45− epithelial cell, or any combination thereof, in a sample obtained from the subject.

In some embodiments, the sample derived, obtained, or isolated from the subject comprises a tissue selected from: mLN, LP of the SI, IEL of the SI, or any combination thereof.

In some embodiments, the determining step is performed in the subject or in a sample derived or obtained from the subject. In some embodiments, the sample comprises any bodily fluid, cell, tissue, biopsy, organ, or a combination thereof, derived or obtained from the subject. In some embodiments, the determining step is performed in vitro, ex vivo, or in vivo. In some embodiments, ex vivo or in vitro comprises or is in a test tube or in a plate.

Methods for determining increased activity, abundance, or both, of cells as disclosed herein, are common and would be apparent to one of ordinary skill in the art. Non-limiting example of such a method includes, but is not limited to, an immunoassay, such as flow cytometry, enzyme-linked immunosorbent assay (ELISA), and others, using specific antibodies targeted to the herein disclosed identifying markers, e.g., CD45, CD4, CD8, CD, Foxp3, CD25, CD11b, CD11c, and others as aforementioned.

A person of ordinary skill in the art can determine TGF-β signaling by measuring for example TGF-β gene and/or protein production, and activation/inhibition of molecules from the TGF-β TGF-β receptor signaling pathway. Non-limiting example of such tests, include, but are not limited to: measure of the level of phosphorylation of SMAD2, which can be performed, for example by an immunoassay such as western-blot, quantify gene expression levels by PCR amplification, e.g., real-time RT-PCR, or next generation sequencing, and protein quantification e.g., by MS/MS or western-blot (followed by densitometry).

One of skill in the art would appreciate that the human mid insula is located in coordinates: (10(−10)) mm anterior-posterior; ±(30-37) mm medial-lateral; and (20 (−15)) mm dorsal-ventral.

In some embodiments, determining the specific coordinates in mpIC, e.g., so as to activate or inhibit the GI, DI, or both, or AI, respectively, in order to maintain or promote, or impair oral tolerance, respectively, can be performed in a murine model. A skilled artisan appreciate that murine neural coordinates can be converted to human neural coordinates.

In some embodiments, activating neurons of the GI, DI, or both in murine model organism, comprises applying NIBS to the following coordinates: (0.14-0.18) mm anterior-posterior; (3.6-3.8) mm medial-lateral; and (2.5-2.6) mm dorsal-ventral.

In some embodiments, inhibiting comprises applying NIBS to the following coordinates: (0.14-0.18) mm anterior-posterior; (3.8-4.0) mm medial-lateral; and (2.7-2.8) mm dorsal-ventral.

In some embodiments, the subject is afflicted or at risk of developing an immune-associated disease. In some embodiments, the subject is afflicted or at risk of developing an autoimmune disease. In some embodiments, risk comprises high risk. In some embodiments, high risk comprises 50% or more.

In some embodiments, the immune-associated disease comprises a food-induced immune disease. In some embodiments, a food-induced immune disease comprises a food allergy. In some embodiments, the immune-associated disease is an autoimmune disease.

In another embodiment, the disease is an immune system disorder. In another embodiment, an immune system disorder is associated with an abnormal overactivity of the immune system. In cases of immune system overactivity, the body attacks and damages its own tissues (autoimmune diseases). In another embodiment, the disease is an immune deficiency disease. In another embodiment, the disease is an allergy. In another embodiment, the disease is an inflammatory or an autoinflammatory disease.

In some embodiments, the disease comprises an inflammatory bowel disease (IBD). In some embodiments, IBD comprises Crohn's disease or ulcerative colitis. In some embodiments, the disease comprises irritable bowel syndrome (IBS).

In some embodiments, the present invention is directed to a method for inducing an immune response in a subject in need thereof, the method comprising: (a) activating neurons of the agranular insula of the subject, (b) inhibiting neurons of the: dysgranular insula of the subject, granular insula of the subject, or both, or (a) and (b).

In some embodiments, an immune response is selected from: vaccination response, humoral response, cytotoxic response, innate immune response, acquired immune response, or any combination thereof. In some embodiments, the subject is afflicted with an immunodeficient disease. In some embodiments, the subject is afflicted with an infectious disease. In some embodiments, the infectious disease is a viral disease. In some embodiments, the subject is afflicted with cancer. In some embodiments, the subject is in need of vaccination.

As used herein, the term “subject” refers to any subject for whom therapy is desired. In another embodiment, a subject is a mammal. In another embodiment, a subject is a human subject. In another embodiment, a subject is a farm animal. In another embodiment, a subject is a pet. In another embodiment, a subject is a lab animal. In another embodiment, a subject is a rodent.

As used herein, the terms “increased” or “to increase” is by: at least 5%, at least 20%, at least 50%, at least 75%, at least 100%, at least 250%, at least 500%, at least 750%, or at least 1,000% compared to control, or any value and range therebetween. Each possibility represents a separate embodiment of the invention. In some embodiments, increased is by 5-25%, 20-75%, 50-120%, 75-150%, 100-250%, 200-550%, 500-750%, or 700-1,000% compared to control. Each possibility represents a separate embodiment of the invention.

As used herein, the term “control” encompasses a subject, or a sample derived therefrom, wherein the subject was not applied with NIBS e.g., neurofeedback, magnetic stimulation, or others as disclosed herein, to neurons of the mpIC.

In some embodiments, the control is a response (e.g., an impaired oral tolerance response) without or in the absence of NIBS e.g., neurofeedback, magnetic stimulation application, etc.

One of ordinary skill in the art would appreciate applying neurofeedback as described hereinabove as part of medication or improvement thereof.

As used herein, “neurofeedback” makes available to a subject a record of one or more of the subject's neurological activities to which the subject ordinarily does not have direct conscious access. In general, the invention is directed to a method of training an individual subject to modify his or her neuronal activity during neurofeedback sessions that utilize real time brain imaging or recording. In some embodiments, the method comprises a step of providing feedback to the subject to enable the subject to modify his or her neuronal activity within the selected brain region, regions, or circuits. In some embodiments, the selected brain target being imaged is associated with a specific disease or disorder, as provided herein.

In one embodiment, the method of the present invention is directed to fMRI-based neurofeedback. fMRI measures blood oxygen level dependent (BOLD) T2* weighted signal changes as an indirect way of visualizing neuronal activity in a localized brain area. The terms “fMRI feedback”, “fMRI neurofeedback”, and the like, are interchangeable, and refer herein to the use of a fMRI device to display or provide a representation of a subject's brain activity to the subject in a real-time or substantially simultaneous manner.

In one embodiment, neurofeedback is an EEG (electroencephalogram) neurofeedback. As used herein, “EEG neurofeedback” refers to a subject's EEG activity as the physiological system that is used for neurofeedback. In another embodiment, an EEG waveform vary in frequency of 0.01 to 100 Hz. In another embodiment, an EEG is recorded from an electrode sensor placed on or in the brain. In another embodiment, EEG is recorded from an electrode sensor placed on the scalp surface. In another embodiment, in EEG neurofeedback the brain wave profile is presented to the subject and the subject is rewarded for changing the profile. In another embodiment, a reward includes, but not limited to, a pleasant-sounding tone, a continuous tone, a dichotomous tone, a visual display, or others.

In one embodiment, neurofeedback according to the method of the present invention comprises any combination of fMRI, fNIRS, DWI or DW-MRI, fMRS and EEG neurofeedback.

In the description and claims of the present application, each of the verbs, “comprise”, “include” and “have” and conjugates thereof, are used to indicate that the object or objects of the verb are not necessarily a complete listing of components, elements or parts of the subject or subjects of the verb.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

In the description unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

In the description and claims of the present application, each of the verbs, “comprise”, “include” and “have” and conjugates thereof, are used to indicate that the object or objects of the verb are not necessarily a complete listing of components, elements or parts of the subject or subjects of the verb.

Other terms as used herein are meant to be defined by their well-known meanings in the art.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

### EXAMPLES

Generally, the nomenclature used herein, and the laboratory procedures utilized in the present invention include chemical, molecular, biochemical, and cell biology techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); “Cell Biology: A Laboratory Handbook”, Volumes Cellis, J. E., ed. (1994); The Organic Chemistry of Biological Pathways by John McMurry and Tadhg Begley (Roberts and Company, 2005); Organic Chemistry of Enzyme-Catalyzed Reactions by Richard Silverman (Academic Press, 2002); Organic Chemistry (6th Edition) by Leroy “Skip” G Wade; Organic Chemistry by T. W. Graham Solomons and, Craig Fryhle.

### Example 1

### Neurons in the mpIC Respond Oral Consumption of Ovalbumin (OVA)

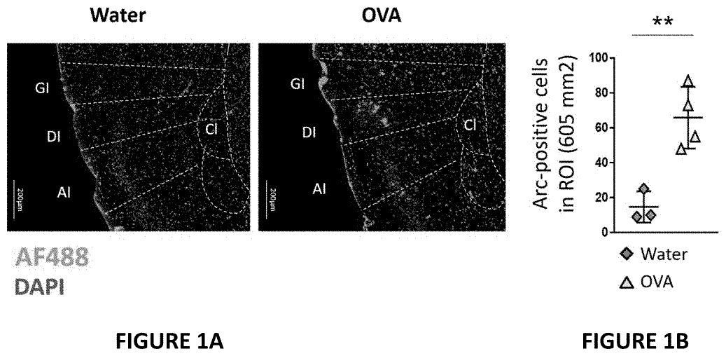

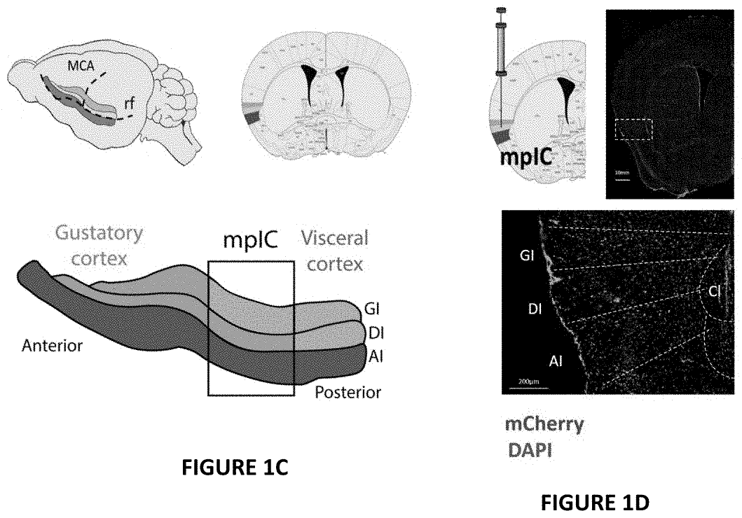

Using transgenic reporter mice that express a fluorescent marker (dVenus) under the control of the endogenous Arc promoter, an immediate early gene (IEG) that expressed in neurons following exposure to salient stimuli, the inventors found that neurons in the mid-posterior region of the IC (mpIC; FIG. 1C) respond to oral consumption of OVA, but not water (FIGS. 1A-1B).

To determine whether antigen-induced neural activity in the mpIC can modulate the immune response towards ingested antigens and thus to affect oral tolerance, the inventors used designer receptors exclusively activated by designer drugs (DREADDs) that allowed to control neuronal activity at will (FIG. 1D). The inventors expressed the activating or inhibiting form of DREADDs (Gi) under the promoter of synapsin by stereotactic injection of a viral vector.

### Example 2

### Inhibition of Neuronal Activity in the mpIC Impairs Oral Tolerance

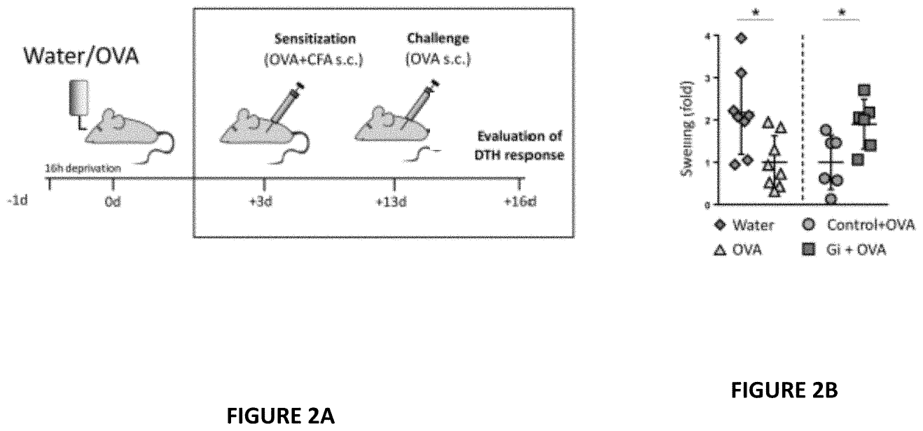

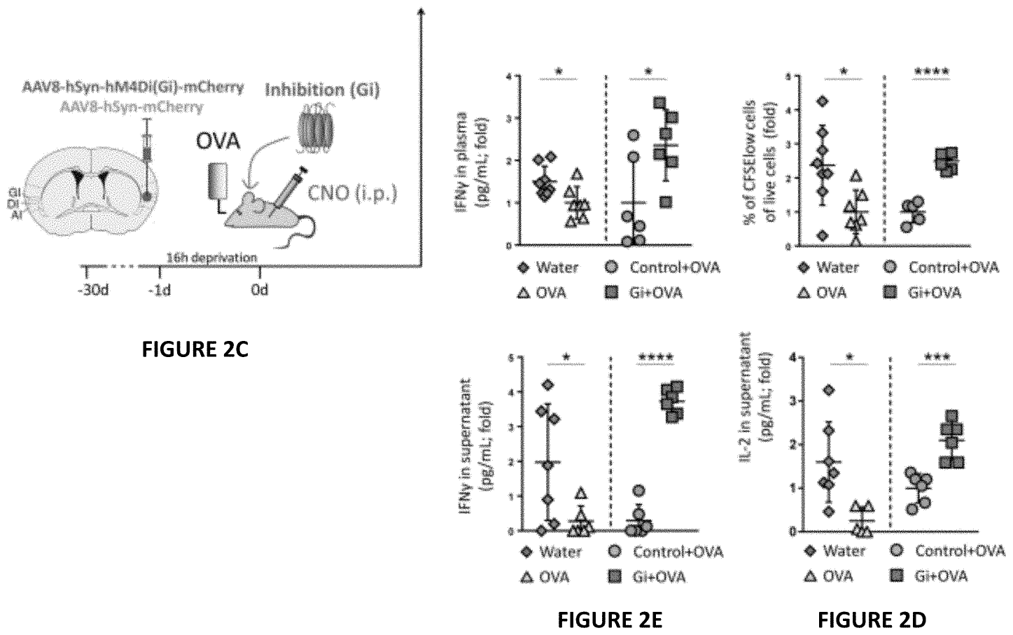

The inventors performed a first experiment, as follows: the delayed-type hypersensitivity model (DTH) is described. This model allowed evaluating oral tolerance as mice that consume OVA prior to the sensitization with OVA in this model already develop antigen-specific regulatory T cells at the time of challenge with OVA, thus the DTH response is suppressed. However, mice that consume water prior to the sensitization develop the DTH response. Second experiment performed was as follows: mice were subjected to the same model, just this time both groups were exposed to OVA and received an i.p. injection of Clozapine-N-Oxide (CNO). The CNO inhibits neuronal activity only in the experimental group, as the control mice were injected with a control virus that did not contain the information for the DREADD (FIG. 2A).

The inventors demonstrated that the mpIC-inhibited mice developed a DTH response even though they consumed OVA before the sensitization and had more resemblance to mice that consume water in this model (FIGS. 2B-2E).

Taken together, these results demonstrated that inhibition of neuronal activity in the mpIC elicited by oral exposure to novel antigen results in impaired tolerance and a DTH response to a subsequent challenge with the antigen.

### Example 3

### Inhibition of Neuronal Activity the mpIC Affects Immune Cells in the Gut and mLNs

The inventors had inhibited the mpIC while the mice were exposed to OVA as before, just this time the immune response in the mesenteric lymph nodes (mLNs) and both compartments of the small intestine (SI), the lamina propria (LP) and the intraepithelial layer (IEL) was examined 24 hours following inhibition.

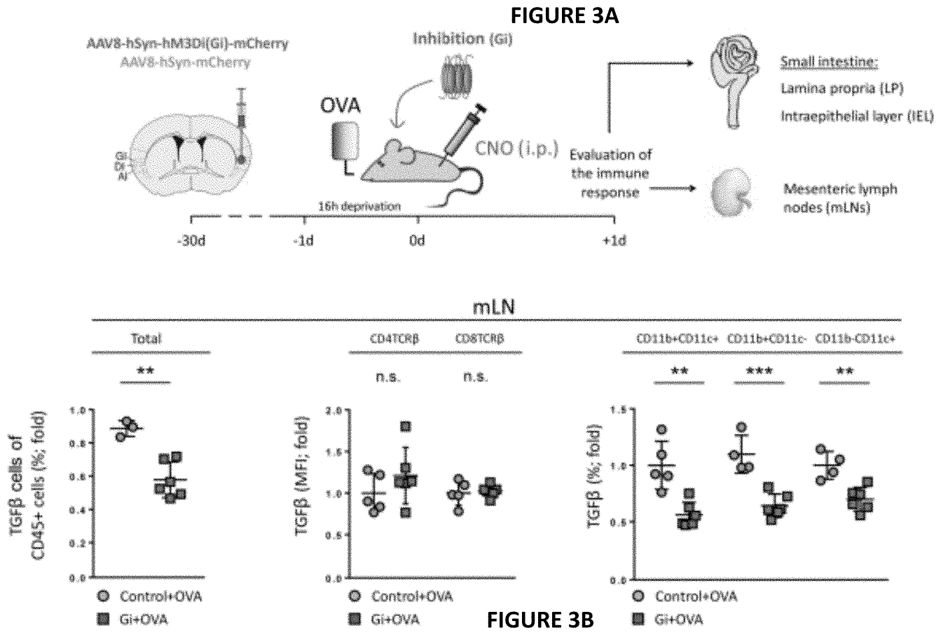

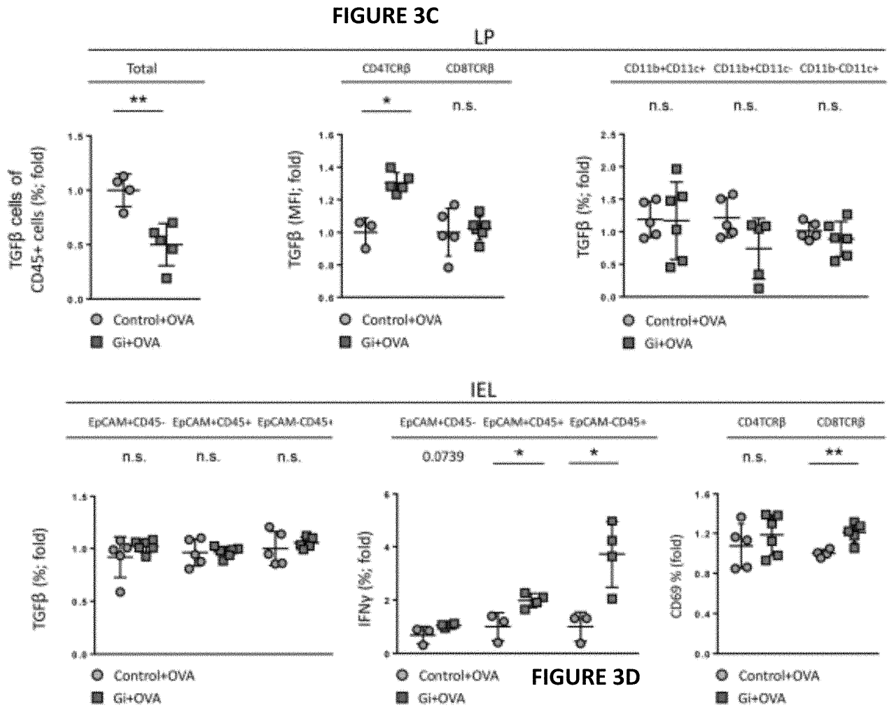

The proportion of TGF-β-expressing immune cells (CD45+) in the draining mLNs was significantly reduced by this manipulation (FIG. 3B: Left panel).

Although T cells appeared to be the main source of TGF-β in the body, no change could be detected in the level of TGF-β expression on CD4+ or CD8+ T cells (FIG. 3B; Middle panel). Nonetheless, the inventors found a substantial reduction in the percent of various TGF-β-expressing myeloid cell populations in the mpIC-inhibited mice compared to control (FIG. 3B; Right panel).

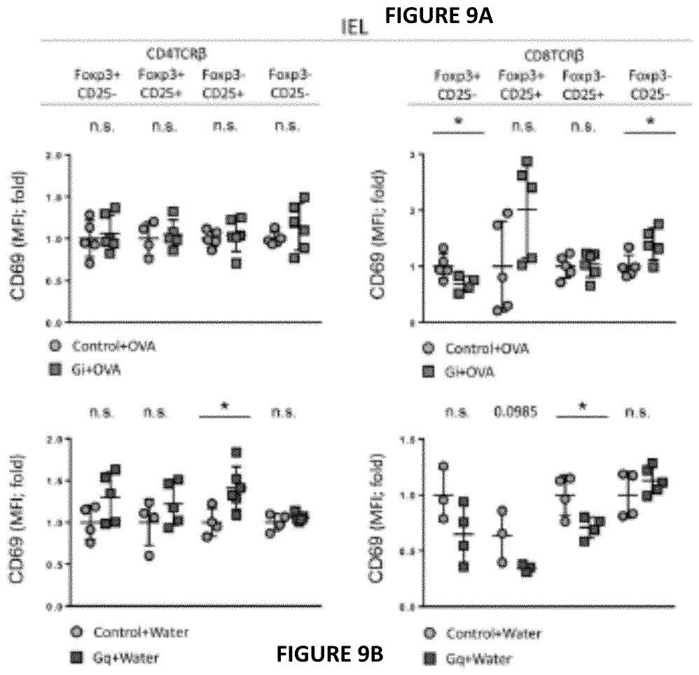

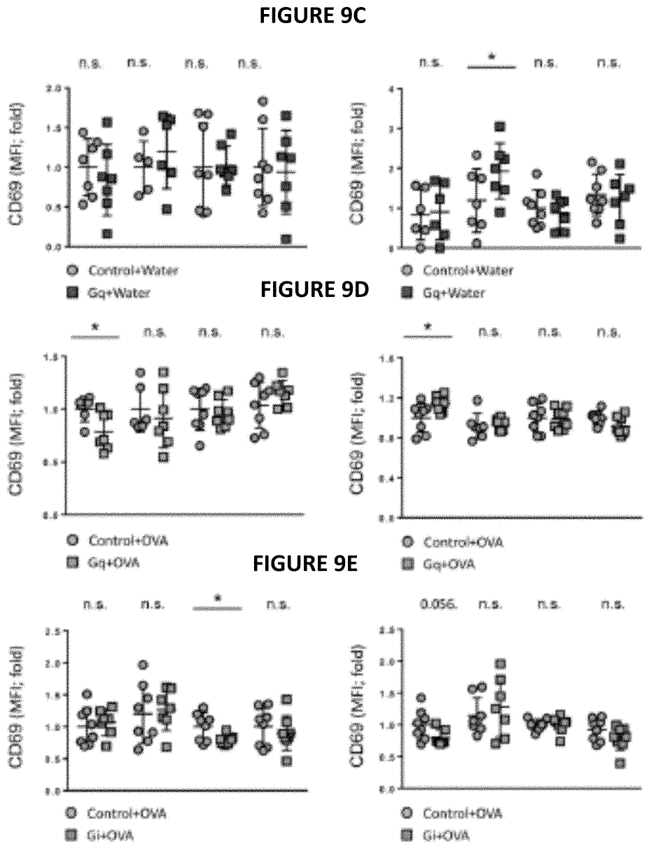

Similarly to the mLN, the inhibition of the mpIC caused a decrease in the proportion of TGF-β-expressing immune cells (CD45+) in LP of the SI (FIG. 3C; Left panel). However, nor T cells or myeloid cells showed a significant reduction in TGF-β-expressing cells, and the inventors did not detect any other specific immune cell subpopulation in the LP that was responsible for this effect (FIG. 3C; Middle and Right panels). Another rich source of TGF-β in the gut is epithelial cells that are located in the IEL and are known to play a major role in the establishment of a tolerogenic environment in the gut. Nevertheless, no change in TGF-β production could be detected in the epithelial cells (EpCAM+CD45+/−) or immune cell populations that are embedded between them (EpCAM-CD45+; FIG. 3D; Left panel). Nonetheless, the data showed elevated expression of IFNγ on immune cells and CD45-expressing epithelial cells in the IEL of mpIC-inhibited mice compared to control (FIG. 3D; middle panel). Priming of IFNγ production appears to be a characteristic feature of the early mucosal immune response to an antigen. In the gut, IFNγ was previously associated with increased gut permeability, development of food allergies and inflammatory bowel disease (IBD). Accordingly, the inventors provide data showing an increased proportion of activated CD69-expressing T cells in the IEL of the mpIC-inhibited mice (FIG. 3D; Right panel right). Further analysis indicated that the expression of CD69 was elevated on CD4+ and CD8+ non-regulatory T cells (Foxp3-CD25-), while it was slightly decreased on CD8+ regulatory cells (Foxp3+CD25-; FIG. 9A).

Collectively, these results suggest the possibility that food-related sensory information acquired by the mpIC can be involved in the maintenance of a tolerogenic environment in the intestine and mLNs, as the lack of this activity seems to cause alternations in regulatory mechanisms that induce the tolerogenic response to innocuous ingested antigens.

### Example 4

### Boosting Oral Tolerance Through Activation of Neurons in the mpIC

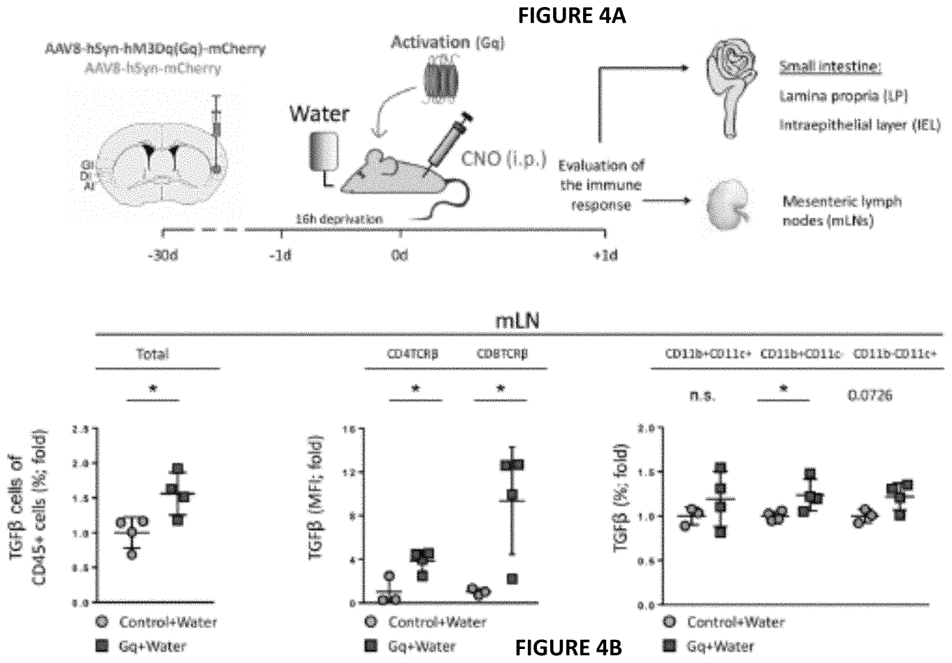

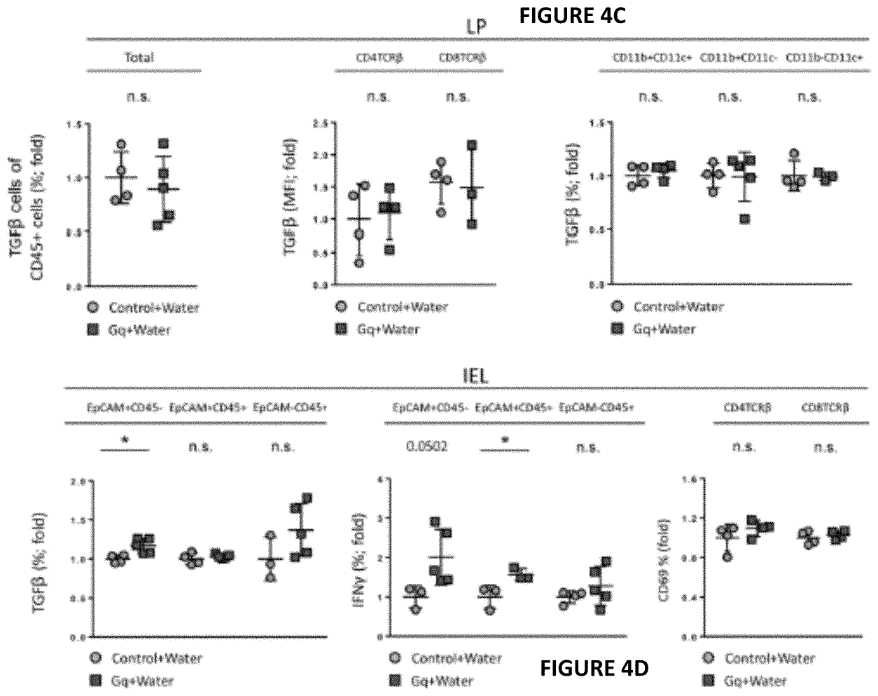

The aforementioned results suggest that OVA-driven activity in the mpIC can induce a state in the gut that promotes oral tolerance. To test this possibility, the inventors stereotactically injected the activating form of DREADDs (Gq) and activated the mpIC while mice were exposed to water and evaluated the immune response in the gut 24 hours following activation. The results demonstrated that activation of neurons in the mpIC results in an elevated proportion of TGFβ-expressing immune cells (CD45+) in the mLNs (FIG. 4B; Left panel).

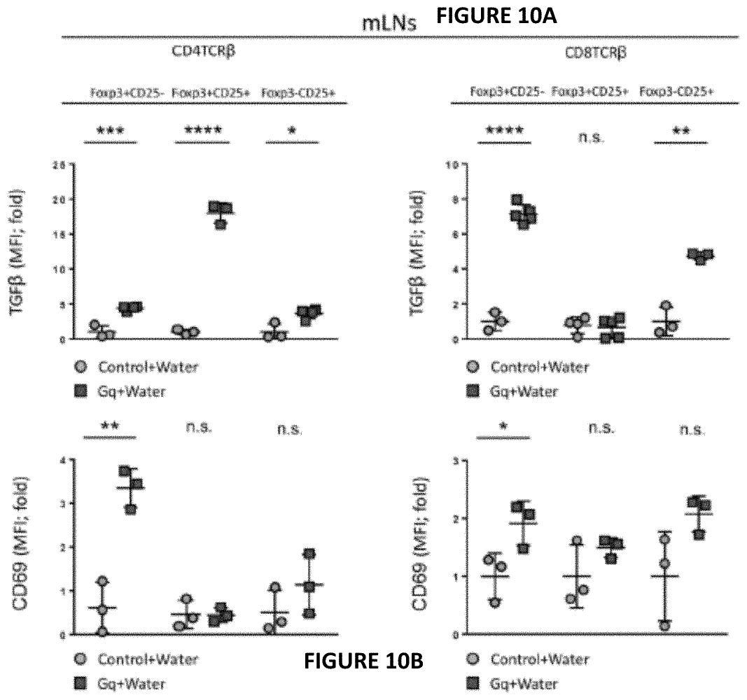

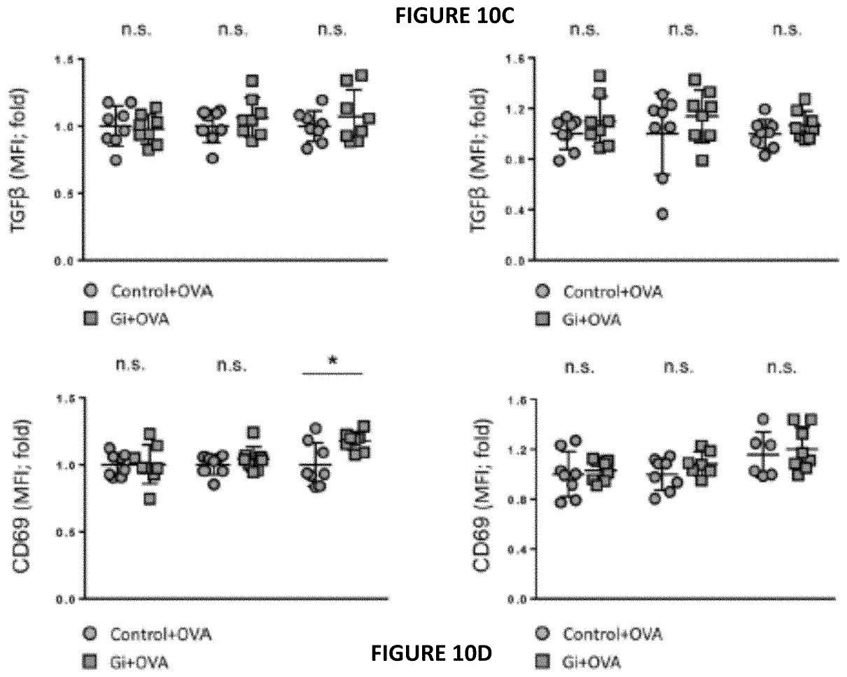

Cell types that were affected by this manipulation were different. Specifically, the percent of TGF-β-expressing CD4+ and CD8+ T cells in the mLNs of the mpIC-activated mice was substantially increased (FIG. 4B; Middle panel). Further analysis showed that the activation caused an increase of almost 20-fold and 8-fold in the proportion of TGF-β-positive Foxp3+CD25+CD4TCRβ and Foxp3+CD25-CD8TCRβ T cells, respectively (FIG. 10A).

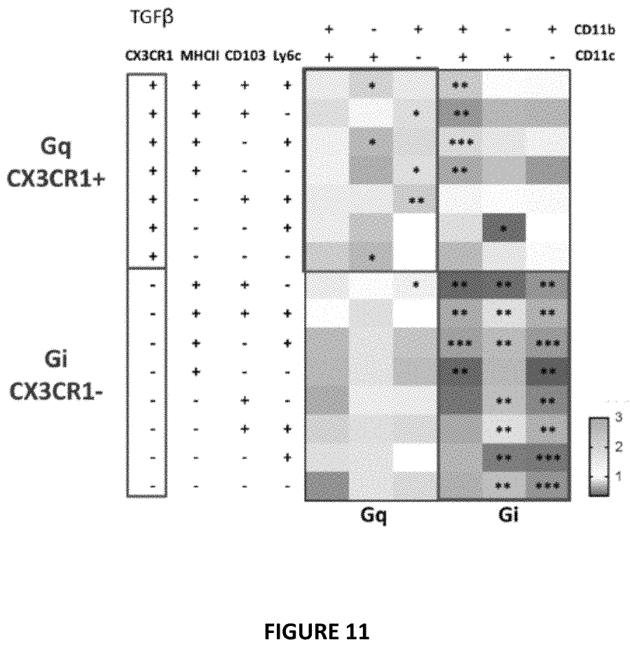

The inventors found a higher proportion of CD11b+CD11c− myeloid cells that expressed TGF-β in the mLN of the mpIC-activated mice (FIG. 4B; Right panel). Further analysis of TGF-β-expressing immune subpopulations showed that activation of the mpIC while mice consume water affected mainly CX3CR1-positive myeloid cells in mLNs. In contrast, inhibition of the mpIC while mice consume OVA had a broader effect on myeloid cells in the mLN and also affected CX3CR1-negative cells (FIG. 11).

The inventors did not detect any changes in TGF-β-producing cells in the lamina propria (LP) following the manipulation (FIG. 4C).

A higher percentage of epithelial cells that produced TGF-β and IFNγ (EpCAM+CD45− and EpCAM+CD45+) were found in the IEL of mpIC-activated mice (FIG. 4D; Left and Middle panels).

A general change in CD69 expression on CD4+ or CD8+ T cells in the IEL was not observed (FIG. 4D; Right panel). However, the data showed that the percent of Foxp3-CD25+CD8+ activated cells was lower in the mpIC-activated mice (FIG. 9B).

Collectively, the results suggest that appetitive sensory signals integrated by the mpIC in the brain can induce a tolerogenic state in the gut and mLNs, while the inhibition of this activity may impair tolerance. The effects are mediated through the modulation of immune subsets and mediators that play a role in the establishment of oral tolerance and suggest that the brain by itself can regulate this process.

### Example 5

### Representation of Aversive Information in the mpIC

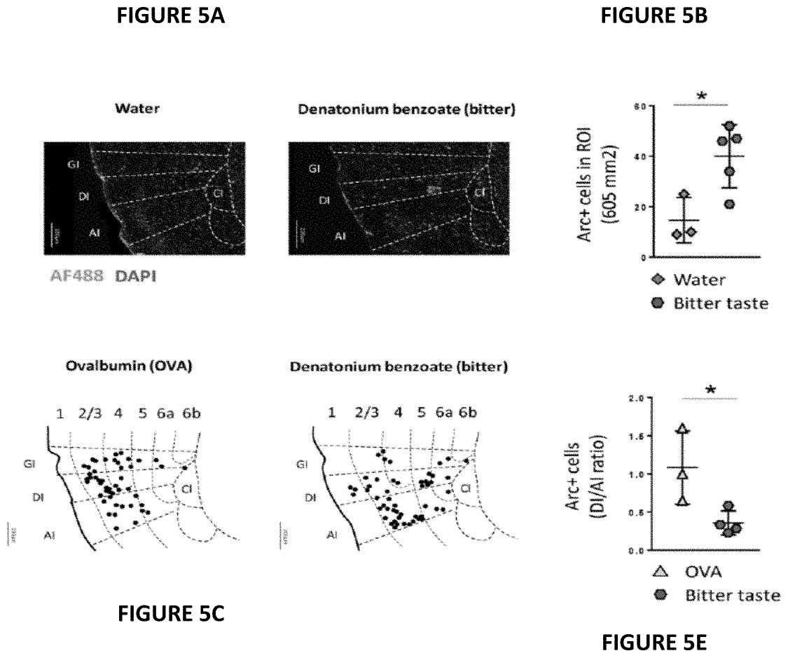

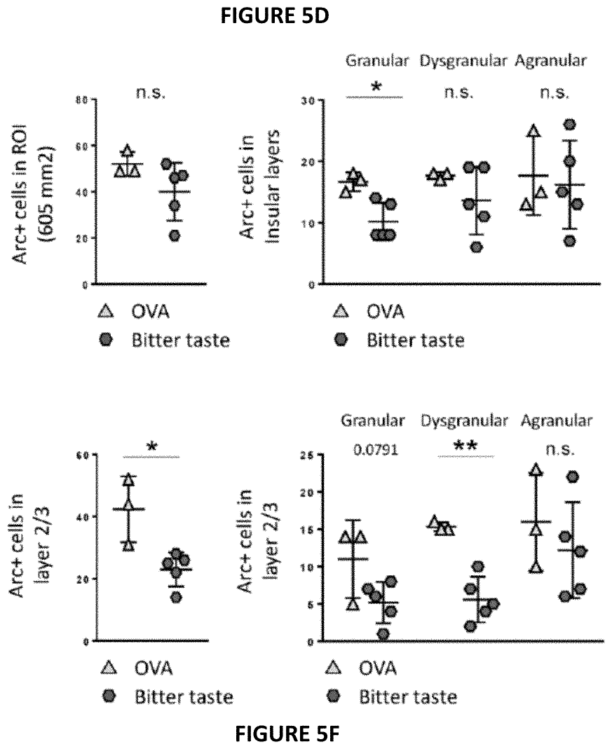

Thus far, the inventors exposed the mice only to positive appetitive signals, however, whether the mpIC also responds to aversive information that can inform about the presence of pathogens, left unclear. To answer this question the inventors used the Arc reporter mice, except this time they were exposed to the bitter substance denatonium benzoate. Analysis of Arc-expressing cells in these mice showed that neurons in the mpIC also respond to aversive information, such as a bitter taste (FIGS. 5A-5B).

When the neural response to denatonium benzoate was compared to the response to OVA, the inventors found that although there was no significant difference in the total number of activated neurons in the mpIC (FIG. 5C), the mice that were exposed to bitter taste expressed less Arc-positive cells in the granular layer (GI) of the mpIC, comparing to mice that consumed OVA (FIG. 5D).

Moreover, the ratio between the activated cells in the dysgranular layer (DI) and agranular layer (AI) of the mpIC was found to be lower in the mice that were exposed to bitter (FIG. 5E).

Further analysis showed that the decrease in Arc-positive neurons in mice that were exposed to bitter was mainly to less Arc-positive cells in the ⅔ layer of the cerebral cortex (FIG. 5F).

### Example 6

### Activation of the Agranular Layer of mpIC Results in Anti-Tolerogenic Effects

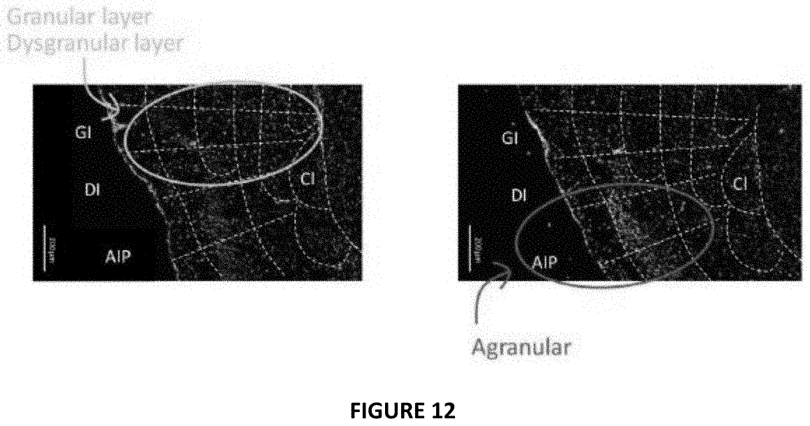

The aforementioned experiments, focused on appetitive positive sensory signals, wherein the inventors targeted the granular (GI) and dysgranular (DI) layers of the mpIC, the layers that receive gustatory and visceral information. However, to explore the effect of aversive sensory information integrated by the mpIC on the immune system the inventors decided to target the agranular (AI) layer of the mpIC (FIG. 12). This decision was based on the analysis of Arc-expression described above, indicating that mice bitter have less activated cells in the GI and DI layers of the mpIC comparing to mice that consume OVA. Moreover, accordingly to the literature, the neurons in the AI of the IC communicate with limbic areas (e.g., amygdala and lateral hypothalamus) that are involved in the processing of aversive stimuli. Accordingly, the AI itself is active following exposure to different aversive stimuli.

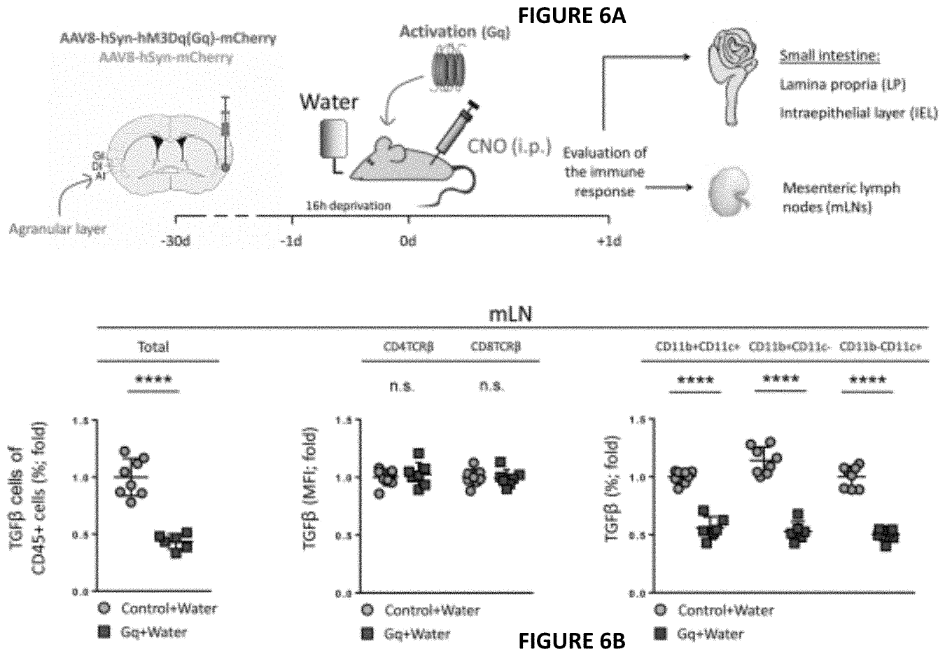

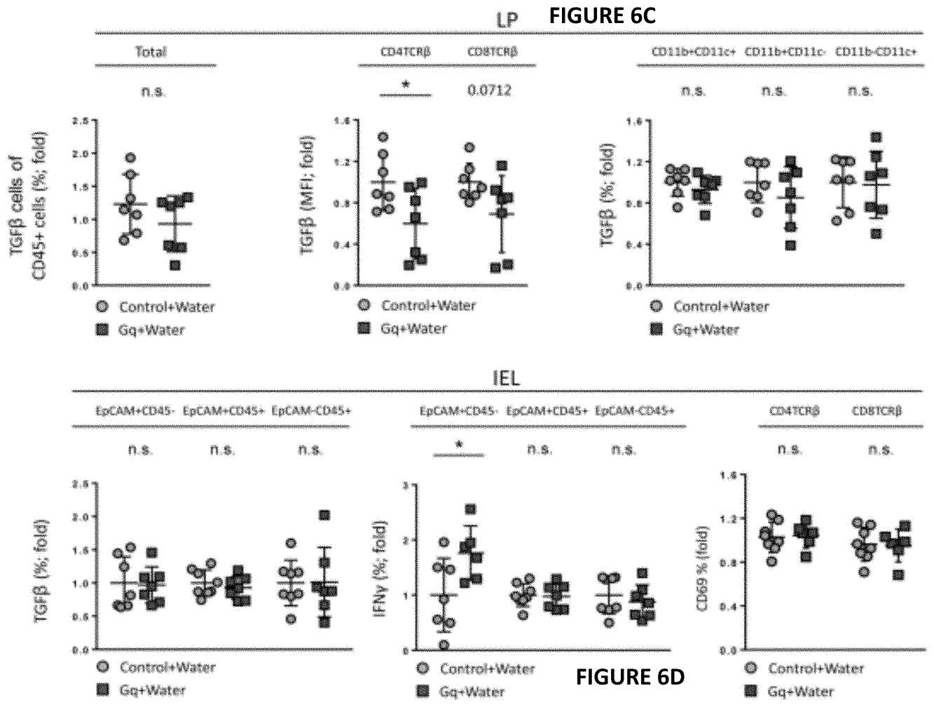

The inventors targeted the AI layer of the mpIC, by injecting the activating form of DREADDs more laterally and ventrally, relatively to previous experiments (FIG. 12). Then, following recovery, the inventors exposed the mice to water while activating the AI layer of mpIC by CNO injection and evaluated the immune response in the gut and mLNs 24 hours later as before (FIG. 6A).

The inventors hypothesized that the activation of the AI layer of the mpIC will affect immune cells in the gut in a similar way to the inhibition of OVA-driven activity in the GI and DI layers of the mpIC that was described earlier, and thus will impair tolerance development.

Indeed, activation of the AI layer of the mpIC caused a reduction in the TGF-β-expressing immune cells in the mLNs (FIG. 6B; Left panel). The data showed that the cells that were responsible for this effect belong to the myeloid progeny, just like in the experiment where the inventors inhibited OVA-driven activity in the GI- and DI layer of the mpIC (FIG. 6B; Right panel).

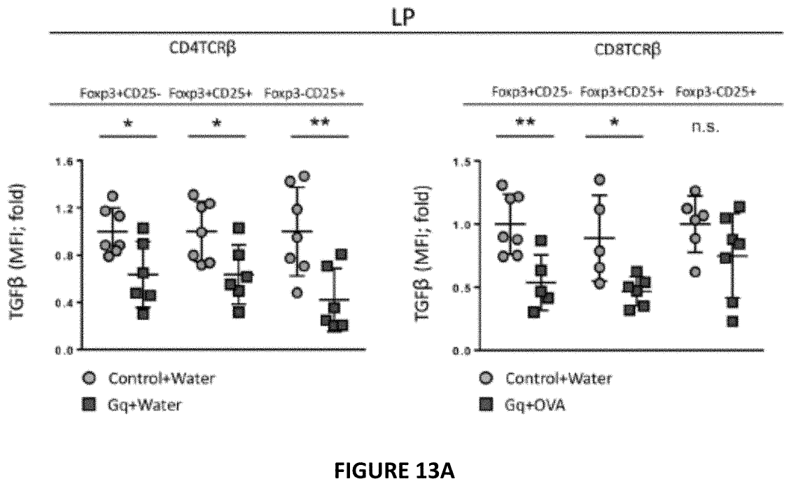

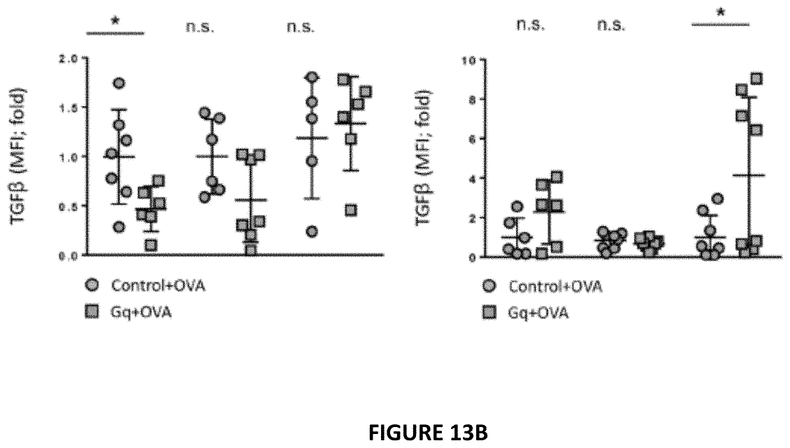

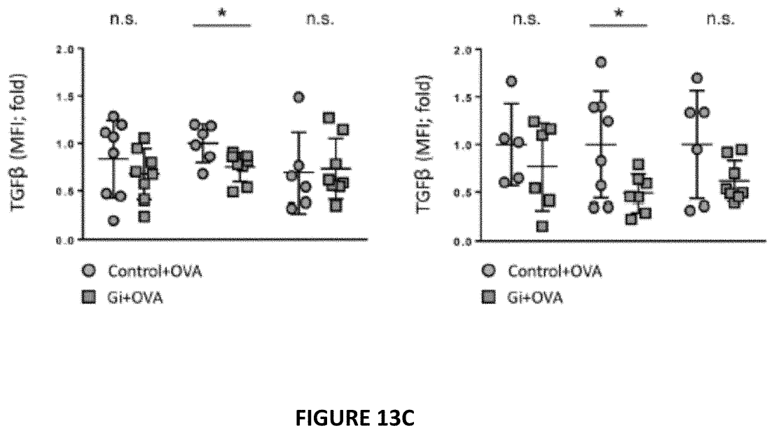

The inventors could not detect a significant change in TGF-β on the total immune cell population (CD45+) in the LP (FIG. 6C; Left panel). Nonetheless, the data demonstrated a reduction in almost all analyzed TGF-β-expressing CD4+ and CD8+ T cell populations of AI-mpIC-activated mice (FIG. 6C; Middle panel), comparing to the control group (FIG. 13A). A significant change in TGF-β in the IEL was not detected (FIG. 6D; Left panel), but there was a higher percent of epithelial cells (EpCAM+CD45) that produce IFNγ in the AI layer of the mpIC activated mice (FIG. 6D; Center panel). Any change in the expression of the activation marker CD69 on T cells in the IEL was not detected (FIG. 6D; Right panel). However, the inventors found a trend towards a reduction in the percent of Foxp3+CD25+ activated (CD69+) CD8TCRβ T cells in the IEL of AI-mpIC activated mice (FIG. 9C).

These results suggest that activation of the agranular layer (AI) of mpIC by aversive sensory stimuli can impair the development of oral tolerance to a novel antigen by modulation of tolerance-mediating cells and factors.

### Example 7

### Activation of the AI Layer of the mpIC During the Exposure to Novel Antigen Results in Anti-Tolerogenic Effects

The similarity of the effects between activation of the AI layer and inhibition of OVA-driven activity in the GI and DI layers of the mpIC suggests that OVA-driven activity in the GI and DI layers can be inhibited as a result of AI-activation when an aversive stimulus is present. To test this possibility, the inventors activated the AI layer of the mpIC while the mice were consuming OVA.

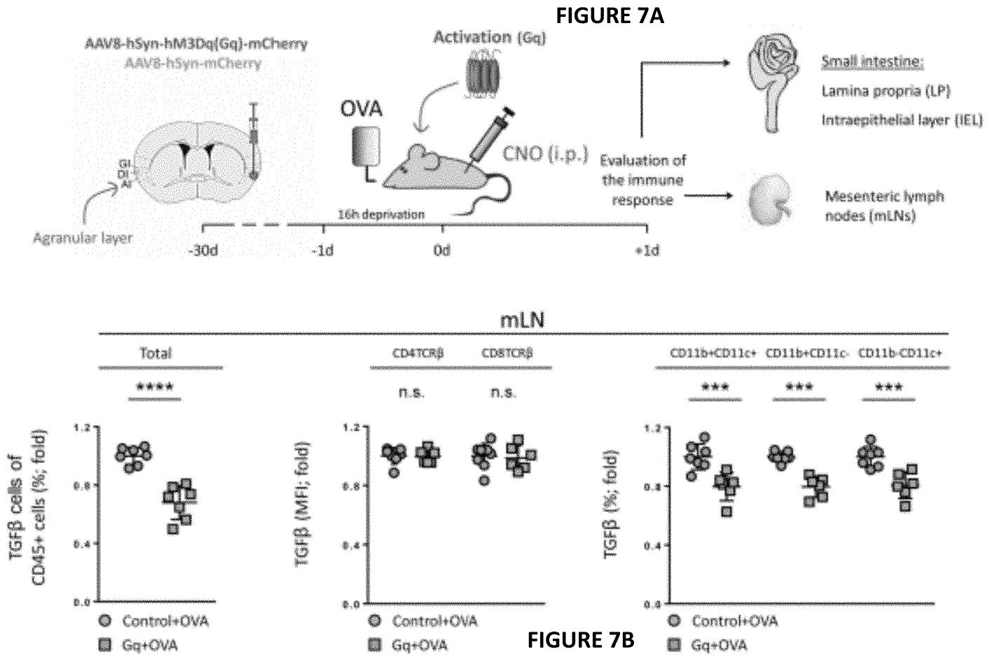

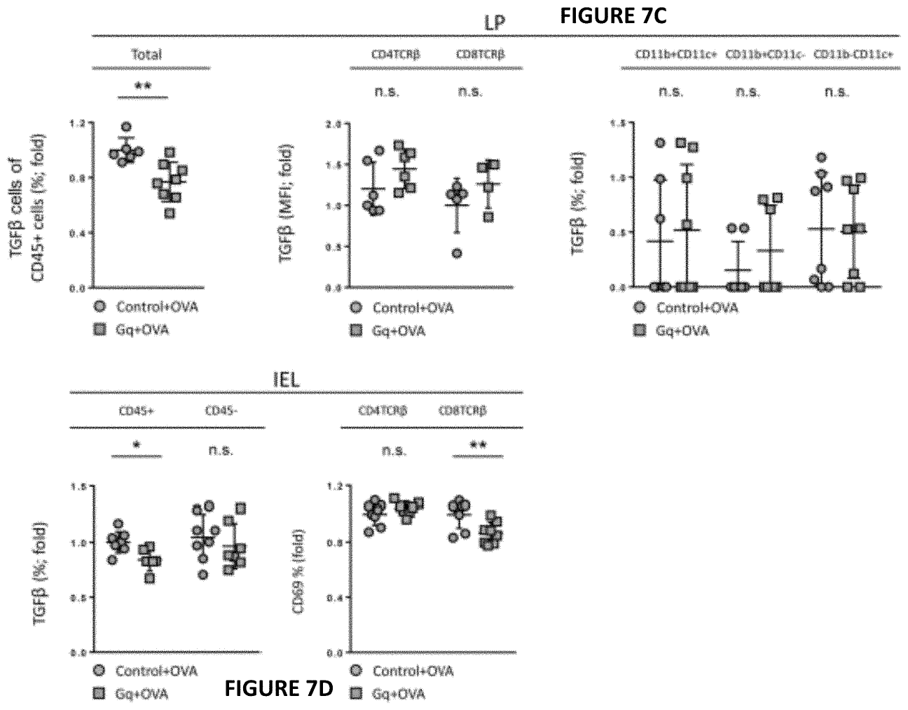

24 hours following ingestion and activation of the AI layer of the mpIC, the percent of TGF-β-expressing cells was reduced in the mLNs and LP (FIGS. 7B-7C). The cells that were responsible for the effect in the mLNs were from the myeloid progeny. In the LP, the percent of TGF-β-expressing Foxp3+CD25-CD4+ T cells was decreased, while the proportion of Foxp3-CD25+CD8+ T cells that produce TGF-β was increased (FIG. 13B).

In the IEL, the activation resulted in a decrease in TGF-β-expressing non-immune cells (CD45-) and in activated (CD69+) CD8TCRβ T cells (FIG. 7D). Further analysis showed that the reduction was in Foxp3+CD25− CD8+ T cells. Moreover, the inventors observed an increase in activated Foxp3+CD25− CD4+ cells (FIG. 9D).

Altogether, these data suggest that activation of the AI layer of the mpIC by aversive stimuli such a bitter taste while ingesting an antigen can reduce tolerance mediating factors and thus impair the development of tolerance and to activate pro-inflammation. As the inventors found less activated cells in the GI and DI layers of the mpIC of Arc-dVenus reporter mice that consume bitter taste comparing to OVA, it is possible that the effects of AI-activation are mediated in the brain by the inhibition of neuronal activity in the GI and DI layers.

### Example 8

### Boosting Oral Tolerance Through Inhibition of Neurons in the AI Layer of the mpIC

When analyzing Arc expression in the Arc-dVenus reporter mice, the inventors observed that although OVA-consuming mice showed lower expression in the AI layer of the mpIC comparing to mice that were exposed to denatonium benzoate, all OVA-consuming mice did express Arc to some extent in the AI layer, and some of them showed a more prominent activation of this layer. This variation in AI activity can represent the variation in AI activity in humans. For example, IBD patients had been shown to have a hyperactive AI layer.

As disclosed hereinabove, the AI is connected to limbic areas that can inform about aversive stimuli, such as bitter taste. However, an unpleasant taste is not the only aversive signal that can be present when eating something for the first time; the novelty of food also can serve as aversive stimuli, a phenomenon termed “food neophobia”. It implies both reduced intake of liquids and foods associated with novelty in comparison with later encounters as the food becomes familiar. If no aversive consequences occur following the ingestion, intake increases on later encounters until reaching the asymptote, this being termed attenuation of taste neophobia. The insular cortex was previously found to play a role in novelty, food neophobia, and attenuation of food neophobia. For these reasons, the inventors hypothesized that the neurons in the AI layer of the mpIC of OVA-consuming mice in the aforementioned experiments were activated due to the novelty of the OVA solution. If so, and accordingly to the herein disclosed results showing that activation of AI while ingesting an antigen can reduce tolerogenic factors, inhibition of this novelty-driven activity in the AI layer of the mpIC in OVA-consuming mice may push the system to the other direction and to induce a tolerogenic state.

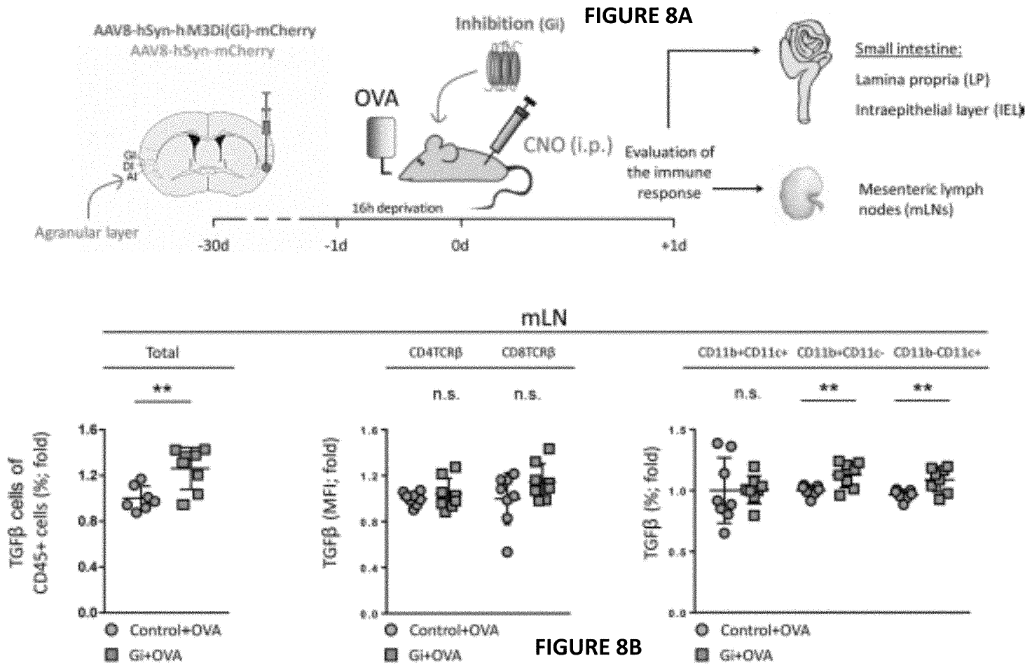

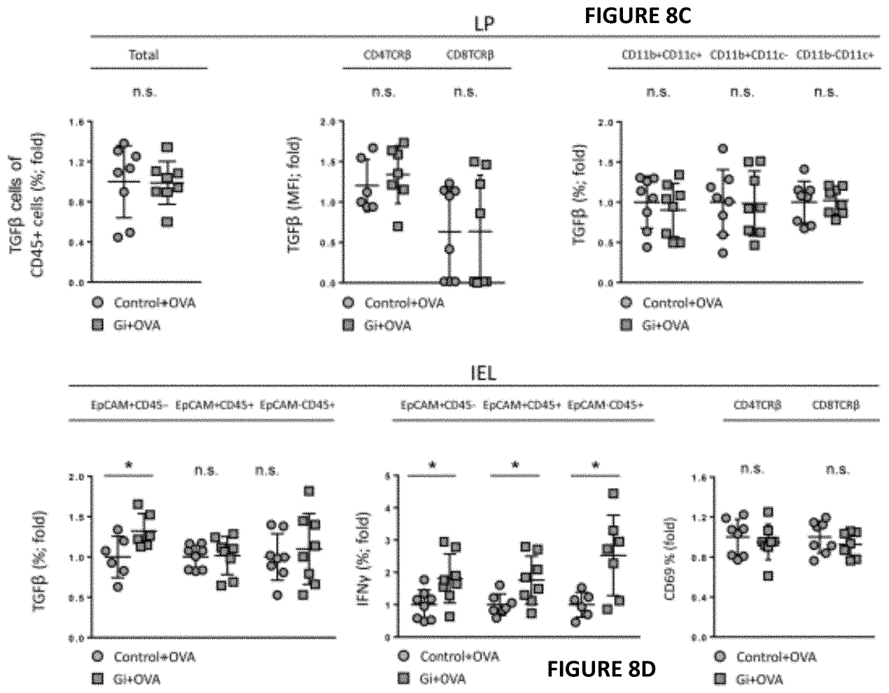

Therefore, the inventors inhibited the AI layer of the mpIC while mice were consuming OVA and evaluated the immune response 24 hours later as before (FIG. 8A).

The inhibition elevated the percent of TGF-β-expressing cells in the mLNs (FIG. 8B). The cells that were responsible for these effects were myeloid cells (CD11b+CD11c+ and CD11b−CD11c+). No significant changes were found in TGF-β expression in the LP (FIG. 8C).

In the IEL, the inventors observed a higher proportion of epithelial cells (EpCAM+CD45-) that expressed TGF-β in the AI-mpIC inhibited mice, comparing to control (FIG. 8D; Left panel). The inventors also observed that a higher percentage of these cells and other cell types in the IEL (EpCAM+CD45+/EpCAM-CD45+) expressed IFNγ (FIG. 8D; Middle panel).

When analyzing the expression of the activation marker CD69, the inventors did not detect any change in the general CD4+ and CD8+ T cell populations (FIG. 8D; Right panel). However, a lower percentage of Foxp3-CD25+CD4TCRb cells was observed in the AI-inhibited mice (FIG. 9).

Taken together, these data suggest that the inhibition of neural activity in the AI layer of the mpIC while being exposed to a novel antigen can promote tolerance in the gut and mLNs. The results support the idea that activation of neurons in the AI layer of the mpIC results in the inhibition of neurons in the GI and DI layers, and consequently, in the impairment of oral tolerance development. These results are interesting from a clinical point of view, as patients with IBD and other immune-related gastrointestinal pathologies show hyperactivity of the AI among other brain regions. The herein disclosed results can explain how and why this hyperactivity is related to the pathology and thus to offer AI-suppression- and/or inhibition-based therapy.

While certain features of the invention have been described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A method for preserving or promoting oral tolerance in a subject in need thereof, comprising the step of modulating neurons in the mid-posterior region of the insular cortex (mpIC) of said subject, thereby preserving or promoting oral tolerance in the subject.

2. The method of claim 1, wherein said modulating comprises:
a. inhibiting neurons of the agranular insula of said subject,
b. activating neurons of the: dysgranular insula of said subject, granular insula of said subject, or both,
or (a) and (b).

3. The method of claim 1, wherein said modulating comprises a step of applying a non-invasive brain stimulation (NIBS) to said subject.

4. The method of claim 3, wherein said NIBS is selected from neurofeedback or magnetic stimulation (MS).

5. The method of claim 1, wherein said preserving or promoting oral tolerance comprises increasing the activity, the abundance, or both, of at least one cell selected from the group consisting of: a CD11b+CD11c+ myeloid cell, CD11b+CD11c− myeloid cell, a CD11b−CD11c+ myeloid cell, a Foxp3+CD25+CD4TCRβ T cell, a Foxp3+CD25+CD8TCRβ T cell, and a EpCAM+CD45− epithelial cell.

6. The method of claim 5, wherein said increased activity, abundance, or both, comprises increased transformation growth factor beta (TGF-β) signaling in said at least one cell.

7. The method of claim 1, wherein said preserving or promoting oral tolerance comprises increasing the number of any one of: (i) TGF-β expressing CD11b+CD11c− myeloid cells, TGF-β expressing CD11b+CD11c+ myeloid cells, TGF-β expressing CD11b−CD11c+ myeloid cells, or any combination thereof; (iii) TGF-β expressing EpCAM+CD45− epithelial cells; (iv) TGF-β expressing Foxp3+CD25+CD4TCRβ cells, TGF-β expressing Foxp3+CD25+CD8TCRβ cells, or both, and any combination of (i) to (iv), in at least one tissue of said subject, wherein said tissue is selected from the group consisting of: mesenteric lymph node (mLN), the lamina propria (LP) of the small intestine, and the intraepithelial layer (IEL) of the small intestine.

8. The method of claim 1, further comprising a step of determining an increased activity, abundance, or both, of at least one cell selected from the group consisting of: a CD11b+CD11c− myeloid cell, a CD11b+CD11c+ myeloid cell, a CD11b−CD11c+ myeloid cell, a Foxp3+CD25+CD4TCRβ cell, a Foxp3+CD25+CD8TCRβ cell, a EpCAM+CD45− epithelial cell, and any combination thereof, in a sample obtained or derived from said subject.

9. The method of claim 1, further comprising a step of determining increased TGF-β signaling in at least one cell selected from the group consisting of: CD11b+CD11c− myeloid cell, CD11b+CD11c+ myeloid cell, CD11b−CD11c+ myeloid cell, Foxp3+CD25+CD4TCRβ cell, Foxp3+CD25+CD8TCRβ cell, EpCAM+CD45− epithelial cell, and any combination thereof, in a sample obtained or derived from said subject.

10. The method of claim 1, wherein said subject is afflicted with an immune-associated disease.

11. The method of claim 10, wherein said immune-associated disease is any one of an autoimmune disease and a food-induced immune disease.

12. A method for increasing TGF-β signaling in a subject in need thereof, comprising a step selected from:
a. inhibiting neurons of the agranular insula of said subject,
b. activating neurons of the: dysgranular insula of said subject, granular insula of said subject, or both, or
c. a combination of (a) and (b);
thereby increasing TGF-β signaling in the subject.

13. The method of claim 12, wherein said increasing TGF-β signaling comprises a step of applying a non-invasive brain stimulation (NIBS) to said subject.

14. The method of claim 13, wherein said NIBS is selected from neurofeedback or magnetic stimulation (MS).

15. The method of claim 12, wherein said increasing TGF-β signaling is in at least one cell selected from the group consisting of: a CD11b+CD11c+ myeloid cell, CD11b+CD11c− myeloid cell, a CD11b−CD11c+ myeloid cell, a Foxp3+CD25+CD4TCRβ T cell, a Foxp3+CD25+CD8TCRβ T cell, and a EpCAM+CD45− epithelial cell.

16. The method of claim 12, wherein said increasing is in at least one tissue of said subject selected from the group consisting of: mesenteric lymph node (mLN), the lamina propria (LP) of the small intestine, and the intraepithelial layer (IEL) of the small intestine.

17. The method of claim 12, wherein said subject is afflicted with an immune-associated disease.

18. The method of claim 17, wherein said immune-associated disease comprises a food-induced immune disease.

19. The method of claim 17, wherein said immune-associated disease comprises an autoimmune disease.

20. The method of claim 19, wherein said autoimmune disease comprises an inflammatory bowel disease (IBD).
